(19) United States
(12) Patent Application Publication
(10) Pub. No.: US 2019/0151414 A1
(43) Pub. Date: May 23, 2019

(54) ANTI-COMPLEMENT FACTOR C4/C4B ANTIBODIES AND USES THEREOF

(71) Applicant: ANNEXON, INC., South San Francisco, CA (US)

(72) Inventors: Ted Yednock, Forest Knolls, CA (US); Sethu Sankaranarayanan, Fremont, CA (US)

(21) Appl. No.: 16/300,207

(22) PCT Filed: May 10, 2017
(86) PCT No.: PCT/US2017/031930

§ 371 Date: Nov. 9, 2018

Related U.S. Application Data
(60) Provisional application No. 62/334,077, filed on May 10, 2016.

(51) Int. Cl. A61K 38/17; A61P 25/28; A61P 29/00

(52) U.S. Cl. CPC A61K 38/1774; A61K 45/06; A61P 29/00; A61P 25/28

(57) ABSTRACT

The present disclosure relates generally to anti-C4 antibodies and anti-C4b antibodies and methods of using them in the treatment of neurodegenerative disease.

### RELATED APPLICATIONS

This Application claims the benefit of U.S. Provisional Application 62/334,077, filed May 10, 2016, which is hereby incorporated by reference in its entirety.

### BACKGROUND

Neurodegenerative diseases are debilitating disorders of the nervous system that affect approximately 30 million individuals worldwide. Neurodegenerative diseases are challenging to treat and are also a growing health concern, both in terms of mortality and the cost of care for the afflicted. The nervous system is a fragile element of the body and has a limited capacity to regenerate from both acute injuries, such as stroke and spinal cord injury, or degenerative diseases. Neurodegenerative diseases can be characterized by progressive loss of neuronal subtypes in the brain and spinal cord and may be either sporadic or familial. Symptoms of neurodegenerative diseases commonly appear during middle or old age. Given the increasing life expectancy of the population, the incidence of these diseases will increase. New therapies are needed to treat neurodegenerative diseases.

### SUMMARY

The present disclosure is generally directed to anti-C4 antibodies and anti-C4b antibodies and uses thereof.

Human complement was originally defined as the heat-labile component of plasma that “complemented” the humoral system and aided antibody-dependent killing of bacteria. Complement is now known to be a tightly regulated proteolytic network of more than 30 proteins circulating in the blood or attached to membrane surfaces that coordinate crucial roles in mammalian innate immunity, especially as it relates to inflammation and the body's defense against invading organisms. Complement proteins are produced by many cell types and have diverse cooperative functions. For example, complement is involved in the clearance of self-antigens and apoptotic cells, forms a bridge to adaptive immunity, and also plays a significant role in tissue regeneration and tumor growth. To exercise these functions, the complement system relies on an interplay of soluble and cell-surface-bound proteins that interact with pathogen cell surfaces to mark them for destruction by phagocytes. The complement system is made up of a large number of distinct plasma proteins, primarily produced by the liver. A number of these proteins are a class of proteases known as zymogens, which are themselves activated by proteolytic cleavage. These zymogens may be widely distributed in an inactive form until an invading pathogen is detected. The complement system thus is activated through a triggered enzyme cascade.

Complement activation is initiated through three pathways: classical, alternative and lectin pathways. All three pathways are initiated by detection of surface structures by pattern recognition proteins. In addition, all three pathways merge through a common intersection, complement C3. C3 is an acute phase reactant. The liver is the main site of synthesis, although small amounts are also produced by activated monocytes and macrophages. A single chain precursor (pro-C3) of approximately 200 kD is found intracellularly; the cDNA shows that it comprises 1,663 amino acids. This is processed by proteolytic cleavage into alpha and beta subunits, which in the mature protein are linked by disulfide bonds. Pro-C3 contains a signal peptide of 22 amino acid residues, the beta chain (645 residues) and the alpha chain (992 residues). The 2 chains are joined by 4 arginine residues that are not present in the mature protein.

The classical pathway is activated by the binding of the complement protein C1q directly to patches of surface-bound antibodies (IgM and IgG), and also to C-reactive protein, serum amyloid P, pentraxin 3, and other known and unknown binding sites on the surfaces of cells, synapses, and microbes.

The lectin pathway is activated by the binding of mannose-binding lectin (MBL) in association with two serum serine proteases designated MASP-1 and MASP-2. MBL is an acute phase protein and its function in the complement pathway is similar to C1q, which it resembles in structure. After MBL binds to the carbohydrate surface of a cell or pathogen, MASP-1 and MASP-2 bind to MBL, and this association causes cleavage and activation of C4 and C2. MASP-1 and MASP-2 proteins are structurally similar to C1r and C1s, and mimic their activities. Similar to the classical complement pathway, the lectin complement pathway also requires C4 and C2 for activation of C3 and other terminal components further downstream in the cascade.

Activation of the complement pathway generates biologically active fragments of complement proteins, e.g., C3a, C4a and C5a anaphylatoxins and sC5b-9 membrane attack complex (MAC), which mediates inflammatory activities involving leukocyte chemotaxis, activation of macrophages, neutrophils, platelets, mast cells and endothelial cells, increased vascular permeability, cytolysis, and tissue injury. Antibody bound to a cell surface antigen can also activate the complement system, creating pores in the membrane of a foreign cell, or it can mediate cell destruction by antibody-dependent cell-mediated cytotoxicity (ADCC). In this process, cytotoxic cells with Fc receptors bind to the Fc region of antibodies on target cells and promote killing of the cells. Antibody bound to a foreign cell also can serve as an opsonin, enabling phagocytic cells with Fc or C3b receptors to bind and phagocytose the antibody-coated cell.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂). Binding of the C1q complex to the surface of a cell or to the complement binding domain of an antibody Fc region induces a conformational change in C1q that leads to activation of an autocatalytic enzymatic activity in C1r, which then cleaves C1s to generate an active serine protease. Once activated, C1s cleaves C4 resulting in C4b, which in turn binds to C2. C2 is cleaved by C1s, resulting in the activated form, C2a, bound to C4b and forming the C3 convertase (C4b2a) of the classical pathway. Ultimately, this pathway leads to the formation of a membrane attack complex, which lyses and kills the affected cell. C2 is a single-chain plasma protein of molecular weight of 102 kD, which is specific for the classical and the lectin complement pathways. Membrane bound C4b expresses a binding site which, in the presence of Mg²⁺, binds the proenzyme C2 near its amino terminus and presents it for cleavage by C1s (for the classical complement pathway) or MASP-2 (for the lectin complement pathway) to yield a 30 kD amino-terminal fragment, C2b, and a 70 kD carboxy-terminal fragment, C2a. The C2b fragment may be released or remain loosely attached to C4b. The C2a fragment consists of a serine protease (SP) and a von Willebrand factor type A (vWFA) domain and remains attached to C4b to form the C4b2a complex, the catalytic components of the C3 and C5 convertases of the classical and the lectin complement pathways. The enzymatic activity in this complex resides entirely in C2a, C4b acting to tether C2a to the activating surface.

C3 is the central component of each complement pathway and is critical for the complement system in both innate and adaptive immune responses. C3b is one of the main effector molecules of the complement system, and cleavage of C3b between the amino acid Cys(⁹⁸⁸) and Glu(⁹⁹¹) results in the release of a highly reactive thioester, which enables C3b to bind to cell surfaces via transacetylation (numbering according to mature protein sequence without signal peptide). Furthermore, cleavage of additional binding sites allows C3b to interact with several regulatory and/or complementary proteins comprising binding sites for CR1 (CD35) or Factor H, both co-factors for cleavage by the protease Factor I. Factor I cleaves C3b between Arg⁽¹²⁸¹⁾ and Ser⁽¹²⁸²⁾ and Arg⁽¹²⁹⁸⁾ and Ser⁽¹²⁹⁹⁾, whereby the fragments C3f and C3bi are generated. C3bi is able to remain attached to the surface of pathogens, where it is recognized by CR3 (CD11b/CD18), which is expressed on macrophages and killer cells. Subsequently, CR3 mediates the phagocytosis and destruction of pathogens. In conjunction with CR1, Factor I can additionally cleave between amino acids Arg⁽⁹³²⁾ and Glu⁽⁹³³⁾, thereby forming C3dg and C3c. C3dg is also capable of remaining on the surface for recognition by CR2 (CD21), which is expressed on B-lymphocytes and dendritic cells (DCs).

C4 is a ˜200 kDa three-chain glycoprotein present in plasma at a concentration of approximately 350 ug/ml. C4 functions as the second complement protein in the classical complement pathway activation sequence. The binding of an appropriate antibody to a substrate leads to binding and activation of the C1 complex. Activated C1 in turn cleaves C4a from the N-terminal of the C4 alpha chain. Such cleavage exposes an internal thioester, which links amino acids at positions 991 and 994 within the C4d region of the C4 alpha subunit. Upon exposure, this highly reactive group undergoes nucleophilic attack to form a covalent bond with the target substrate. The major fragment of C4, C4b, is covalently bound to the target substrate following cleavage and release of C4a, and acts as a receptor for C2 of the classical pathway. C2 binds to C4b and is cleaved in turn, by active C1 to continue the complement cascade.

Complement is nonspecific in that it can attack both foreign invaders and host cells. Under normal conditions, host cells, including neurons, are protected from potential complement-mediated damage by various fluid-phase and membrane-bound complement regulatory proteins, such as C1 inhibitor (C1-Inh). C1-INH dissociates C1r and C1s from the active C1 complex, which protects host cells from lysis or damage from the membrane attack complex. Other proteins that protect from potential complement-mediated damage include C4b-binding protein (C4BP), factor H (FH), complement receptor 1 (CR1; CD35), complement receptor Ig (CRIg), decay accelerating factor (DAF; CD55), membrane cofactor protein (MCP; CD46), and CD59. However, deficiencies of these components or excessive activation of complement in response to certain pathological conditions can overwhelm this protective mechanism. Such unbalanced activation has been associated with a growing number of diseases and pathological disorders.

For example, various complement components are expressed by neurons and glial cells in vitro and in vivo. While their function in the brain is unknown, the expression of many of these complement proteins is upregulated by serum or inflammatory cytokines after brain injury or during the course of neurodegenerative disease pathology. Astrocytes in culture have been reported to express C1q, C1r, C1s, C4, C2, and C3, as well as the more terminal complement proteins. Neurons have been reported to express C4 and C3. C1q was shown to be expressed in neuronal synapses and to mark these synapses for elimination. See, e.g., U.S. Patent Publication Nos. 2012/0195880 and 2012/328601. While selective synapse loss is an essential aspect of normal brain development (“synaptic pruning”), excessive synapse loss, especially in a mature or aging brain, results in neurodegeneration and cognitive decline. Elevated synaptic complement accumulation contributes to synaptic loss in normal aging and in neurodegenerative disease progression. Conversely, lowering complement expression was neuroprotective. Neurons affected by synapse loss may be central nervous system neurons, or peripheral nervous system neurons.

Neutralizing the activity of complement factors such as C4 and/or the C4b portion of C4 can block complement activation, prevent synapse loss, and slow neurodegenerative disease progression as well as cognitive decline in normal aging. Neurodegenerative diseases involving synapse loss and considered to be amenable to treatments aimed at the neutralization of complement factors such as C4 and/or the C4b portion of C4 include Alzheimer's disease, amyotrophic lateral sclerosis, multiple sclerosis, glaucoma, optic neuritis, spinal muscular atrophy, frontotemporal dementia, Parkinson's disease, Huntington's disease, schizophrenia and the like.

In certain aspects, provided herein are methods of preventing, reducing risk of developing, or treating a neurodegenerative disorder (e.g., Alzheimer's disease, amyotrophic lateral sclerosis, epilepsy, glaucoma, Guillain-Barre syndrome, multiple sclerosis, Neuromyelitis Optica (NMO), optic neuritis, spinal muscular atrophy, frontotemporal dementia, Parkinson's disease, schizophrenia or Huntington's disease), comprising administering an antibody that binds to complement component C4 or the C4b portion of C4.

Disclosed herein is a method of inhibiting synapse loss, comprising administering to a patient suffering from adverse synapse loss an antibody, such as an anti-C4 or anti-C4b antibody. The patient may have suffered synapse loss as a result of a neurodegenerative disorder, central nervous system disorder, or a peripheral nervous system disorder. In some embodiments, the neurodegenerative disorder is Alzheimer's disease, amyotrophic lateral sclerosis, epilepsy, glaucoma, Guillain-Barre syndrome, multiple sclerosis, Neuromyelitis Optica (NMO), optic neuritis, spinal muscular atrophy, frontotemporal dementia, Parkinson's disease, schizophrenia or Huntington's disease. The method may further comprise administration of neural progenitors, or a neurogenesis enhancer. In certain preferred embodiments, the antibody binds to C4 and inhibits complement activation.

The methods of the present disclosure may also be applied to treatment and prevention of neurodegenerative disorders of the eye, such as, for example, macular degenerative diseases, chronic open-angle glaucoma, acute closed angle glaucoma, all stages of age-related macular degeneration (AMD), including dry and wet (non-exudative and exudative) forms (AMD), Geographic atrophy, choroidal neovascularization (CNV), uveitis, diabetic and other ischemia-related retinopathies, endophthalmitis, and other intraocular neovascular diseases, such as diabetic macular edema, pathological myopia, von Hippel-Lindau disease, histoplasmosis of the eye, Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, sarcoidosis, temporal arteritis, polyarteritis nodosa, and multiple sclerosis.

A preferred group of neurodegenerative disorders of the eye include glaucoma, age-related macular degeneration (AMD), including non-exudative (wet) and exudative (dry or atrophic) AMD, choroidal neovascularization (CNV), diabetic retinopathy (DR), optic neuritis, NMO, and endophthalmitis.

Disclosed herein is a method of treating or preventing a disease associated with complement activation in an individual in need of such treatment, the method comprising administering an antibody. In some embodiments, the disease associated with complement activation is a neurodegenerative disorder, and the neurodegenerative disorder is associated with the loss of synapses or nerve connections, such as synapse loss that is dependent on the complement receptor 3 (CR3) or complement receptor CR1. The neurodegenerative disorder may also be associated with pathological activity-dependent synaptic pruning, synapse phagocytosis by microglia, e.g., Alzheimer's disease, amyotrophic lateral sclerosis, multiple sclerosis, glaucoma, myotonic dystrophy, Guillain-Barre syndrome (GBS), Myasthenia Gravis, Bullous Pemphigoid, optic neuritis, spinal muscular atrophy, Down syndrome, Parkinson's disease, frontotemporal dementia, schizophrenia and Huntington's disease.

In some embodiments, the disease associated with complement activation is an inflammatory disease, an autoimmune disease, or metabolic disorder selected from diabetes, obesity, rheumatoid arthritis (RA), acute respiratory distress syndrome (ARDS), remote tissue injury after ischemia and reperfusion, complement activation during cardiopulmonary bypass surgery, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), (AMD-wet), Geographic atrophy choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, dry AMD, sarcoidosis, temporal arteritis, polyarteritis nodosa, multiple sclerosis, allo-transplantation, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis. Preferably, the autoimmune disease is an antibody-mediated autoimmune disease. Antibody-mediated autoimmune diseases typically occur when antibodies react to a self-protein or when immune complexes are formed. Antibodies to self-proteins inhibit normal cellular processes and form immune complexes that activate the complement cascade resulting in inflammation and tissue damage.

The disease associated with complement activation can also be an autoimmune disease selected from myasthenia gravis, Diabetes mellitus type 1, Hashimoto's thyroiditis, Addison's disease, Coeliac disease, Crohn's disease, pernicious anaemia, Pemphigus vulgaris, vitiligo, autoimmune hemolytic anemias, paraneoplastic syndromes, a vasculitis disease, hypocomplementemic urticarial vasculitis (HUV), polymyalgia rheumatica, temporal arteritis, Wegener's granulomatosis, multiple sclerosis, Guillain-Barre syndrome, Myasthenia Gravis, Bullous Pemphigoid, or myositis.

In some embodiments, the antibody is an anti-C4 antibody or an anti-C4b antibody. The antibody may block binding to C4, or the C4b portion of C4, or to one or several components of the complement cascade (e.g., activation of C4 by C1s or C1-like complex, such as MASP-1 and/or MASP-2; C4 binding to C2 or the C2a portion of C2; C4b2a binding to C3b; or C5 binding to C4b2a3b). The antibody may bind different epitopes or regions of complexes in the complement cascade. For example, the antibody may bind to C2a and C4b of the C3 convertase, C2a and C3b of the C5 convertase, or C2a and C4b of the C5 convertase, or C4b, C2a and C3b of the C5 convertase.

Full-length antibodies may be prepared by the use of recombinant DNA engineering techniques. Such engineered versions include those created, for example, from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The DNA encoding the antibody may be prepared by deleting all but the desired portion of the DNA that encodes the full length antibody. DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode antibodies include cells, such as hybridomas, that express the full length antibody. For example, the antibody may be isolated from a host cell that expresses an expression vector that encodes the heavy and/or light chain of the antibody.

Antibody fragments may also be prepared by the use of recombinant DNA engineering techniques involving the manipulation and re-expression of DNA encoding antibody variable and constant regions. Standard molecular biology techniques may be used to modify, add or delete further amino acids or domains as desired. Any alterations to the variable or constant regions are still encompassed by the terms ‘variable’ and ‘constant’ regions as used herein. In some instances, PCR is used to generate an antibody fragment by introducing a stop codon immediately following the codon encoding the interchain cysteine of C_{H}1, such that translation of the C_{H}1 domain stops at the interchain cysteine. Methods for designing suitable PCR primers are well known in the art and the sequences of antibody C_{H}1 domains are readily available. In some embodiments, stop codons may be introduced using site-directed mutagenesis techniques.

The antibody of the present disclosure may be derived from any antibody isotype (“class”) including for example IgG, IgM, IgA, IgD and IgE and subclasses thereof, including for example IgG1, IgG2, IgG3 and IgG4. In certain preferred embodiments, the heavy and light chains of the antibody are from murine IgG1.

The antibodies disclosed herein may also cross the blood brain barrier (BBB). The antibody may activate a BBB receptor-mediated transport system, such as a system that utilizes the insulin receptor, transferrin receptor, leptin receptor, LDL receptor, or IGF receptor. The antibody can be a chimeric antibody with sufficient human sequence that is suitable for administration to a human. The antibody can be glycosylated or nonglycosylated; in some embodiments, the antibody is glycosylated, e.g., in a glycosylation pattern produced by post-translational modification in a CHO cell.

The antibodies of the present disclosure may also be covalently linked to a therapeutic agent, such as an anti-inflammatory protein, neurotherapeutic agent, anti-viral, anti-parasitic, anti-bacterial, endocrine drug, metabolic drug, mitotoxin, chemotherapy drug, or siRNA, for which transport across the BBB is desired. The covalent linkage between the antibody and, for example, the neurotherapeutic agent may be a linkage between any suitable portion of the antibody and the therapeutic agent, as long as it allows the antibody-agent fusion to cross the blood brain barrier and the therapeutic agent to retain a therapeutically useful portion of its activity within the central nervous system. For example, the covalent link may be between one or more light chains of the antibody and the therapeutic agent. In the case of a peptide neurotherapeutic agent (e.g., a neurotrophin such as brain derived neurotrophic factor, BDNF), the peptide can be covalently linked by its carboxy or amino terminus to the carboxy or amino terminus of the light chain (LC) or heavy chain (HC) of the antibody.

Other neurotherapeutic agents that can be linked to antibodies of the present disclosure include a neurotrophin selected from brain derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, or stem cell factor (SCF).

In certain embodiments, the antibody has a dissociation constant (K_{D}) for human C4 that ranges from 10 μM to 20 μM, or 1 μM to less than 10 μM. The antibody may have a dissociation constant (K_{D}) for mouse C4 that ranges from 1 μM to 200 μM.

The antibodies of the present disclosure may also specifically bind and neutralize a biological activity of C4, e.g., 1) C2 binding to C4b, (2) C4 cleavage by C1s, (3) C4 cleavage by MASP-1, (4) C4 cleavage by MASP-2, (5) C4 cleavage by MASP-1 and MASP-2, (6) C2a binding to C4b, (7) C4b binding to or cleavage of C3 (as part of the C3 convertase), or (8) C2a binding to or cleavage of C5 (as part of the C5 convertase). The biological activity may also be (1) neutralization of the classical complement activation pathway, (2) neutralization of the lectin complement activation pathway, (3) neutralization of the alternative pathway activity, (4) neutralization of the membrane attack complex (MAC), (5) activation of antibody and complement dependent cytotoxicity, (6) CH50 hemolysis, (7) synapse loss, (8) B-cell antibody production, (9) dendritic cell maturation, (10) T-cell proliferation, (11) cytokine production, (12) microglia activation, (13) Arthus reaction, (14) phagocytosis of synapses or nerve endings, or (15) activation of complement receptor 3 (CR3/C3) expressing cells.

In some embodiments, CH50 hemolysis comprises human, mouse, and/or rat CH50 hemolysis. In some embodiments, the antibody is capable of neutralizing from at least about 50%, to at least about 95% of CH50 hemolysis. The antibody may also be capable of neutralizing at least 50% of CH50 hemolysis at a dose of less than 150 ng, less than 100 ng, less than 50 ng, or less than 20 ng.

Other in vitro assays to measure complement activity include ELISA assays for the measurement of split products of complement components or complexes that form during complement activation. Complement activation via the classical pathway can be measured by following the levels of C4b, C4b2a, C4a, C4d and C4 in the serum. Activation of the alternative pathway can be measured in an ELISA by assessing the levels of Bb or C3bBbP complexes in circulation. An in vitro antibody-mediated complement activation assay may also be used to evaluate inhibition of C3a production.

An antibody of the present disclosure may be a monoclonal antibody, a polyclonal antibody, a recombinant antibody, a humanized antibody, a chimeric antibody, a multispecific antibody, or an antibody fragment thereof.

The antibodies of the present disclosure may also be an antibody fragment, such as a Fab fragment, a Fab′ fragment, a F(ab′)₂ fragment, a Fv fragment, a diabody, or a single chain antibody molecule.

The antibodies disclosed herein may also be coupled to a labeling group, e.g., an optical label, radioisotope, radionuclide, an enzymatic group, biotinyl group, a nucleic acid, oligonucleotide, enzyme, or a fluorescent label. A labeling group may be coupled to the antibody via a spacer arm of any suitable length to reduce potential steric hindrance. Various methods for labeling proteins are known in the art and can be used to prepare such labeled antibodies.

Various routes of administration are contemplated. In some embodiments, the antibody is injected directly into the eye, e.g., for prevention or treatment of an ocular disease or condition, and may be administered by ocular, intraocular, and/or intravitreal injection, for example. Other methods of administration by also be used, which include but are not limited to, topical, parenteral, subcutaneous, intraperitoneal, intrapulmonary, intrathecal, intranasal, and intralesional administration. Parenteral infusions include intramuscular, intravenous, intraarterial, intraperitoneal, or subcutaneous administration. For treatment of central nervous system conditions, the antibody may be adapted to cross the blood-brain barrier following a non-invasive peripheral route of administration such as intravenous intramuscular, subcutaneous, intraperitoneal, or even oral administration.

The present disclosure also provides a method of detecting synapses in an individual, by a) administering an antibody from any of the preceding embodiments to the subject, wherein the antibody is coupled to a detectable label; (b) detecting the detectable label to measure the amount or location of the antibody in the subject; and (c) comparing the amount or location of the antibody to a reference, wherein the risk of developing a disease associated with complement activation is characterized based on the comparison of the amount of antibody as compared to the reference. For example, the detectable label may comprise a nucleic acid, oligonucleotide, enzyme, radioactive isotope, biotin, or a fluorescent label (e.g., fluorescein, rhodamine, cyanine dyes or BODIPY). The detectable label may be detected using an imaging agent for x-ray, CT, MM, ultrasound, PET and SPECT.

### DETAILED DESCRIPTION

The present application relates to methods of preventing, reducing risk of developing, or treating a neurodegenerative disorder (e.g., Alzheimer's disease, amyotrophic lateral sclerosis, multiple sclerosis, glaucoma, optic neuritis, spinal muscular atrophy, frontotemporal dementia, Parkinson's disease, Huntington's disease, or schizophrenia), comprising administering an antibody that binds to complement component C4 or the C4b portion of C4.

Suitable antibodies include antibodies that bind to complement component C4, as well as antibodies that bind to C4b, or the C4b portion of C4. Such antibodies include monoclonal antibodies and homologues, analogs, and modified or derived forms thereof, including Fab, F(ab′)₂, Fv and single chain antibodies.

Preferred antibodies are monoclonal antibodies, which can be raised by immunizing rodents (e.g., mice, rats, hamsters and guinea pigs) with either (1) native C4b derived from enzymatic digestion of C4 purified from human plasma or serum, or (2) recombinant C4b or its fragments expressed by either eukaryotic or prokaryotic systems. Other animals can be used for immunization, e.g., non-human primates, transgenic mice expressing human immunoglobulins, and severe combined immunodeficient (SCID) mice transplanted with human B-lymphocytes.

Polyclonal and monoclonal antibodies are naturally generated as immunoglobulin (Ig) molecules in the immune system's response to a pathogen. A dominating format with a concentration of 8 mg/ml in human serum, the ˜150-kDa IgG1 molecule is composed of two identical ˜50-kDa heavy chains and two identical ˜25-kDa light chains.

Hybridomas can be generated by conventional procedures by fusing B-lymphocytes from the immunized animals with myeloma cells. In addition, anti-C4 or anti-C4b antibodies can be generated by screening recombinant single-chain Fv or Fab libraries from human B-lymphocytes in a phage-display system. The specificity of the MAbs to human C4 or C4b can be tested by enzyme linked immunosorbent assay (ELISA), Western immunoblotting, or other immunochemical techniques.

The inhibitory activity on complement activation of antibodies identified in the screening process can be assessed by hemolytic assays using either unsensitized rabbit or guinea pig RBCs for the alternative complement pathway, or sensitized chicken or sheep RBCs for the classical complement pathway. Those hybridomas that exhibit an inhibitory activity specific for the classical complement pathway are cloned by limiting dilution. The antibodies are purified for characterization for specificity to human C4 or C4b by the assays described above.

Based on the molecular structures of the variable regions of the anti-C4 or anti-C4b antibodies, molecular modeling and rational molecular design may be used to generate and screen small molecules that mimic the molecular structures of the binding region of the antibodies and inhibit the activities of C4 or C4b. These small molecules can be peptides, peptidomimetics, oligonucleotides, or organic compounds. The mimicking molecules can be used as inhibitors of complement activation in inflammatory indications and autoimmune diseases. Alternatively, one can use large-scale screening procedures commonly used in the field to isolate suitable small molecules from libraries of combinatorial compounds.

A suitable exposure of an antibody as disclosed herein may be between 10 and 500 μg/ml of serum. The actual serum exposure can be determined in clinical trials following the conventional methodology for determining optimal dosages, i.e., administering various dosages and determining which doses provide suitable efficacy without undesirable side-effects.

Before the advent of recombinant DNA technology, proteolytic enzymes (proteases) that cleave polypeptide sequences were used to dissect the structure of antibody molecules and to determine which parts of the molecule are responsible for its various functions. Limited digestion with the protease papain cleaves antibody molecules into three fragments. Two fragments, known as Fab fragments, are identical and contain the antigen-binding activity. The Fab fragments correspond to the two identical arms of the antibody molecule, each of which consists of a complete light chain paired with the V_{H} and C_{H}1 domains of a heavy chain. The other fragment contains no antigen binding activity but was originally observed to crystallize readily, and for this reason was named the Fc fragment (Fragment crystallizable).

A Fab molecule is an artificial ˜50-kDa fragment of the Ig molecule with a heavy chain lacking constant domains C_{H}2 and C_{H}3. Two heterophilic (V_{L}-V_{H} and C_{L}-C_{H}1) domain interactions underlie the two-chain structure of the Fab molecule, which is further stabilized by a disulfide bridge between C_{L} and C_{H}1. Fab and IgG have identical antigen binding sites formed by six complementarity-determining regions (CDRs), three each from V_{L} and V_{H} (LCDR1, LCDR2, LCDR3 and HCDR1, HCDR2, HCDR3). The CDRs define the hypervariable antigen binding site of antibodies. The highest sequence variation is found in LCDR3 and HCDR3, which in natural immune systems are generated by the rearrangement of V_{L} and J_{L} genes or V_{H}, D_{H} and J_{H} genes, respectively. LCDR3 and HCDR3 typically form the core of the antigen binding site. The conserved regions that connect and display the six CDRs are referred to as framework regions. In the three-dimensional structure of the variable domain, the framework regions form a sandwich of two opposing antiparallel β-sheets that are linked by hypervariable CDR loops on the outside and by a conserved disulfide bridge on the inside.

Methods are disclosed herein for protecting or treating an individual suffering from adverse effects of synapse loss. Immature astrocytes in normal development produce a signal that induces neurons to express a specific complement protein, thus enabling a developmental window during which synapse elimination occurs. Expression of such a protein in development mirrors the period of developmental synaptogenesis, being off in embryonic brain and adult brain but on at high levels in postnatal brain, when synaptic pruning and elimination occurs.

These findings have broad implications for a variety of clinical conditions, particularly neurodegenerative conditions where synapse loss is involved. Synapse loss is inhibited by contacting neurons with inhibitors or antagonists of the complement pathway. For example, inhibitors can block activation of the complement cascade, can block the expression of specific complement proteins in neurons, can interfere with signaling molecules that induce complement activation, can upregulate expression of complement inhibitors in neurons, and otherwise interfere with the role of complement in synapse loss. The ability to prevent synapse loss, e.g., in adult brains, has important implications for maintaining normal neuronal function in a variety of neurodegenerative conditions.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

The term “about” as used herein refers to the usual error range for the respective value readily known to the skilled person in this technical field. Reference to “about” a value or parameter herein includes (and describes) embodiments that are directed to that value or parameter per se.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” molecule or cell is a molecule or a cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature. Isolated molecules therefore are distinguished from molecules existing naturally in cells; isolated cells are distinguished from cells existing naturally in tissues, organs, or individuals. In some embodiments, the isolated molecule is an anti-C4 antibody of the present disclosure. In other embodiments, the isolated cell is a host cell or hybridoma cell producing an anti-C4 antibody of the present disclosure.

An “isolated” antibody is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In certain preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. An isolated antibody includes the antibody in situ within recombinant T-cells since at least one component of the antibody's natural environment will not be present. Ordinarily, however, an isolated polypeptide or antibody will be prepared by a process including at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

As used herein, the term “CDR” or “complementarily determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table (X) as a comparison. The CDRs listed in Table (X) were defined in accordance with Kabat 1991.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous in that they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein, Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (See, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

“Functional fragments” of antibodies comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO/1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE′ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Leski J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³M⁻¹ or 10⁴M⁻¹, sometimes about 10⁵M⁻¹ or 10⁶M⁻¹, in other instances about 10⁶M⁻¹ or 10⁷M⁻¹, about 10⁸M⁻¹ to 10⁹M⁻¹, or about 10¹⁰ M⁻¹ to 10¹¹M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

Antibody “effector functions” refer to those biological activities attributable to the Fc region (a native sequence Fc region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-C4 antibody binds specifically to an epitope within a complement C4 protein. “Specific binding” refers to binding with an affinity of at least about 10⁻⁷M or greater, e.g., 5×10⁻⁷M, 10⁻⁸ M, 5×10⁻⁸M, and greater. “Non-specific binding” refers to binding with an affinity of less than about 10⁻⁷ M, e.g., binding with an affinity of 10⁻⁶ M, 10⁻⁵M, 10⁻⁴M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

An “isolated” nucleic acid molecule is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acids encoding any polypeptides and antibodies herein that exist naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors useful in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “neurotrophins” refers to neurotrophic factors that are neuroprotective in the brain. These factors are suitable for use in the compositions and methods of the disclosure and include brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-α, TGF-β, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), saposins, semaphorins, and stem cell factor (SCF).

The term “preventing” is art-recognized, and when used in relation to a condition, such as an epilepsy disease, is well understood in the art, and includes administration of a composition which reduces the frequency or severity, or delays the onset, of one or more symptoms of the medical condition in a subject relative to a subject who does not receive the composition. Thus, the prevention of epilepsy disease progression includes, for example, slowing the average amount of neurodegeneration in a population of patients receiving a therapy relative to a control population that did not receive the therapy, e.g., by a statistically and/or clinically significant amount. Similarly, the prevention of neurodegenerative disease progression includes reducing the likelihood that a patient receiving a therapy will develop a disability, such as cognitive decline and/or memory loss, or delaying the onset of disability, relative to a patient who does not receive the therapy.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

As used herein, administration “conjointly” with another compound or composition includes simultaneous administration and/or administration at different times. Conjoint administration also encompasses administration as a co-formulation or administration as separate compositions, including at different dosing frequencies or intervals, and using the same route of administration or different routes of administration.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Nucleic Acids, Vectors and Host Cells

Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-C4 or anti-C4b antibody. In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the V_{L}/C_{L} of the antibody and a second vector containing a nucleic acid that encodes an amino acid sequence containing the V_{H}/C_{H}1 of the antibody. In some embodiments, the host cell is eukaryotic, e.g., a Chinese Hamster Ovary (CHO) cell or lymphoid cell (e.g., Y0, NS0, Sp20 cell).

Methods of making an anti-C4 antibody or an anti-C4b antibody of the present disclosure are provided. In some embodiments, the method includes culturing a host cell of the present disclosure containing a nucleic acid encoding the anti-C4 antibody or anti-C4b antibody, under conditions suitable for expression of the antibody. In some embodiments, the antibody is subsequently recovered from the host cell (or host cell culture medium).

For recombinant production of a humanized anti-C4 or anti-C4b antibody of the present disclosure, a nucleic acid encoding the antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the antibodies of the present disclosure, or fragments thereof, include without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-C4 or anti-C4b antibody of the present disclosure.

Suitable host cells for cloning or expression of antibody-encoding vectors include prokaryotic or eukaryotic cells. For example, an anti-C4 or anti-C4b antibody of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

### Antibody Screening

Candidate antibodies can be screened for the ability to modulate synapse loss. Such screening may be performed using an in vitro model, a genetically altered cell or animal, or a purified protein. A wide variety of assays may be used for this purpose. In some embodiments, antibodies are tested in an in vitro culture system. For example, antibodies may be tested in an in vitro culture system, which includes the addition of microglial cells to cultures of cortical neurons, followed by counting the number of synapses removed from the neurons and/or ingested by the microglial cells.

Functional activity of a candidate antibody may also be tested in vivo by assessing the ability of the antibody to modulate synapse loss during normal aging, e.g., in animals challenged with intracerebral injection of amyloid-beta oligomers, or in animals genetically modified with human familial mutations associated with Alzheimer's disease, Huntington's disease, Parkinson's disease, frontotemporal dementia, spinal muscular atrophy, or in animals prone to spontaneous glaucoma, or in animals with induced forms of glaucoma.

Candidate antibodies may also be identified using computer-based modeling, by binding assays, and the like. Various in vitro models may be used to determine whether an antibody binds to, or otherwise affects complement activity. Such candidate antibodies may be tested by contacting neurons in an environment permissive for synapse loss. Such antibodies may be further tested in an in vivo model for an effect on synapse loss.

Synapse loss may be quantitated by administering the candidate antibodies to neurons in culture, and determining the presence of synapses in the absence or presence of the antibodies. In some embodiments of the disclosure, the neurons are a primary culture, e.g., of retinal ganglion cells (RGCs). Purified populations of RGCs are obtained by conventional methods, such as sequential immunopanning. The cells are cultured in suitable medium, which may comprise appropriate growth factors, e.g., CNTF; BDNF; etc. The neural cells, e.g., RGCs, are cultured for a period of time sufficient to allow robust process outgrowth and then cultured with candidate antibodies for a period of about 1 day to 1 week. In some embodiments, the neurons are cultured on a live astrocyte cell feeder in order to induce signaling for synapse loss. Methods of culturing astrocyte feeder layers are known in the art. For example, cortical glia can be plated in a medium that does not allow neurons to survive, with removal of non-adherent cells.

For synapse quantification, cultures are fixed, blocked and washed, then stained with antibodies specific synaptic proteins, e.g., synaptotagmin, etc. and visualized with an appropriate reagent, as known in the art. Analysis of the staining may be performed microscopically. In some embodiments, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Number, mean area, mean minimum and maximum pixel intensities, and mean pixel intensities for all synaptotagmin, PSD-95, and colocalized puncta in the image are recorded and saved to disk for analysis.

Generally, a plurality of assay mixtures is run in parallel with different antibody concentrations to obtain a differential response to the various concentrations. Typically one of these concentrations serves as a negative control, i.e., at zero concentration or below the level of detection.

### Pharmaceutical Compositions and Administration

In certain aspects, the antibodies of the present disclosure may be administered in the form of pharmaceutical compositions.

Therapeutic formulations of an antibody of the disclosure may be prepared for storage by mixing the antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Lipofections or liposomes may also be used to deliver the antibody or antibody fragment into cell, wherein the epitope or smallest fragment which specifically binds to the binding domain of the target protein is preferred.

The antibody may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for parenteral administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The antibodies and compositions of the present disclosure are typically administered by ocular, intraocular, and/or intravitreal injection, and other methods known in the art. Other methods of administration may also be used, which include but are not limited to, topical, parenteral, subcutaneous, intraperitoneal, intrapulmonary, intranasal, and intralesional administration. Parenteral routes of administration include intramuscular, intravenous, intraarterial, intraperitoneal, intrathecal, or subcutaneous administration.

For example, formulations for ocular, intraocular or intravitreal administration may be prepared by methods and using ingredients known in the art. A main factor in efficient treatment is proper penetration through the eye. Unlike diseases of the front of the eye, where drugs may be delivered topically, retinal diseases benefit from a more site-specific approach. Eye drops and ointments rarely penetrate the back of the eye, and the blood-ocular barrier hinders penetration of systemically administered drugs into ocular tissue. Accordingly, usually the method of choice for drug delivery to treat retinal disease, such as AMD and CNV, is direct intravitreal injection. Intravitreal injections are usually repeated at intervals which depend on the patient's condition, and the properties and half-life of the drug delivered. For intraocular (e.g., intravitreal) penetration, usually molecules of smaller size are preferred.

Pharmaceutical compositions may also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, non-immunogenic stabilizers, excipients and the like. The compositions may also include additional substances to approximate physiological conditions, such as pH adjusting and buffering agents, toxicity adjusting agents, wetting agents and detergents.

The composition may also include any of a variety of stabilizing agents, such as an antioxidant for example. When the pharmaceutical composition includes a polypeptide, the polypeptide may be complexed with various well-known compounds that enhance the in vivo stability of the polypeptide, or otherwise enhance its pharmacological properties (e.g., increase the half-life of the polypeptide, reduce its toxicity, enhance other pharmacokinetic and/or pharmacodynamic characteristics, or enhance solubility or uptake). Examples of such modifications or complexing agents include sulfate, gluconate, citrate and phosphate. The polypeptides of a composition may also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration may be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it may be expressed as the ratio LD50/ED50. Compounds that exhibit large therapeutic indices are preferred.

The data obtained from cell culture and/or animal studies may be used in formulating a range of dosages for humans. The dosage of the active ingredient typically lines within a range of circulating concentrations that include the ED50 with low toxicity. The dosage may vary within this range depending upon the dosage form employed and the route of administration utilized.

The pharmaceutical compositions described herein may be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, intraocular, and intracranial methods.

For oral administration, the active ingredient may be administered in solid dosage forms, such as capsules, tablets, and powders, or in liquid dosage forms, such as elixirs, syrups, and suspensions. The active component(s) may be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents may be used to make compressed tablets. Both tablets and capsules may be manufactured as sustained release products to provide for continuous release of medication over a period of hours. Compressed tablets may be sugar coated or film coated to mask any unpleasant taste and protect the tablet from the atmosphere, or enteric-coated for selective disintegration in the gastrointestinal tract. Liquid dosage forms for oral administration may contain coloring and flavoring to increase patient acceptance.

Formulations suitable for parenteral administration include aqueous and non-aqueous, isotonic sterile injection solutions, which may contain antioxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for parenteral use are usually sterile. To the extent that a given compound must be synthesized prior to use, the resulting product is typically substantially free of any potentially toxic agents, particularly any endotoxins, which may be present during the synthesis or purification process. Compositions for parental administration are also typically substantially isotonic and made under GMP conditions.

The compositions of the disclosure may be administered using any medically appropriate procedure, e.g., intravascular (intravenous, intraarterial, intracapillary) administration, injection into the cerebrospinal fluid, intravitreal, topical, intracavity or direct injection in the brain. Intrathecal administration may be carried out through the use of an Ommaya reservoir, in accordance with known techniques. (F. Balis et al., Am J. Pediatr. Hematol. Oncol. 11, 74, 76 (1989).

The efficacy regarding the method of treatment of complement-associated eye conditions, such as AMD or CNV, may be measured by various endpoints commonly used in evaluating intraocular diseases. For example, vision loss may be assessed. Vision loss may be evaluated by, but not limited to, e.g., measuring by the mean change in best correction visual acuity (BCVA) from baseline to a desired time point (e.g., where the BCVA is based on Early Treatment Diabetic Retinopathy Study (ETDRS) visual acuity chart and assessment at a test distance of 4 meters), measuring the proportion of subjects who lose fewer than 15 letters in visual acuity at a desired time point compared to baseline, measuring the proportion of subjects who gain greater than or equal to 15 letters in visual acuity at a desired time point compared to baseline, measuring the proportion of subjects with a visual-acuity Snellen equivalent of 20/2000 or worse at a desired time point, measuring the NEI Visual Functioning Questionnaire, measuring the size of CNV and amount of leakage of CNV at a desired time point, e.g., by fluorescein angiography, etc. Ocular assessments may be done, e.g., which include, but are not limited to, e.g., performing eye exam, measuring intraocular pressure, assessing visual acuity, measuring slitlamp pressure, assessing intraocular inflammation, etc.

Where the therapeutic agents are locally administered in the brain, one method for administration of the therapeutic compositions of the disclosure is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition given to a particular patient may depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD50 animal data, and other information, a clinician may determine the maximum safe dose for an individual, depending on the route of administration. Utilizing ordinary skill, the competent clinician will be able to optimize the dosage of a particular therapeutic composition in the course of routine clinical trials. The compositions may be administered to the subject in a series of more than one administration. For therapeutic compositions, regular periodic administration will sometimes be required, or may be desirable. Therapeutic regimens will vary with the agent; for example, some agents may be taken for extended periods of time on a daily or semi-daily basis, while more selective agents may be administered for more defined time courses, e.g., one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc., in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e., having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Kits

The present disclosure also provides a pharmaceutical pack or kit comprising one or more containers filled with one or more of the ingredients of the pharmaceutical compositions. Associated with such container(s) may be a notice in the form prescribed by a governmental agency regulating the manufacture, use or sale of pharmaceuticals or biological products, which notice reflects approval by the agency of manufacture, use or sale for human administration.

Kits of the present disclosure may include one or more containers comprising a purified anti-C4 antibody or anti-C4b antibody and instructions for use in accordance with methods known in the art. Generally, these instructions comprise a description of administration of the inhibitor to treat or diagnose a disease, according to any methods known in the art. The kit may further comprise a description of selecting an individual suitable for treatment based on identifying whether that individual has the disease and the stage of the disease.

The instructions generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating a specific disease. Instructions may be provided for practicing any of the methods described herein.

The kits of this disclosure are preferably disposed in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a mini-pump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an inhibitor of classical complement pathway. The container may further comprise a second pharmaceutically active agent.

Kits may optionally provide additional components such as buffers and interpretive information. Normally, the kit comprises a container and a label or package insert(s) on or associated with the container.

### Inhibition of Complement

A number of molecules are known that inhibit the activity of complement. In addition to known compounds, suitable inhibitors can be screened by methods described herein. As described above, normal cells can produce proteins that block complement activity, e.g., CD59, C1 inhibitor, etc. In some embodiments of the disclosure, complement is inhibited by upregulating expression of genes encoding such polypeptides.

Modifications of molecules that block complement activation are also known in the art. For example, such molecules include, without limitation, modified complement receptors, such as soluble CR1. The mature protein of the most common allotype of CR1 contains 1998 amino acid residues: an extracellular domain of 1930 residues, a transmembrane region of 25 residues, and a cytoplasmic domain of 43 residues. The entire extracellular domain is composed of 30 repeating units referred to as short consensus repeats (SCRs) or complement control protein repeats (CCPRs), each consisting of 60 to 70 amino acid residues. Recent data indicate that C1q binds specifically to human CR1. Thus, CR1 recognizes all three complement opsonins, namely C3b, C4b, and C1q. A soluble version of recombinant human CR1 (sCR1) lacking the transmembrane and cytoplasmic domains has been produced and shown to retain all the known functions of the native CR1. The cardioprotective role of sCR1 in animal models of ischemia/reperfusion injury has been confirmed. Several types of human C1q receptors (C1qR) have been described. These include the ubiquitously distributed 60- to 67-kDa receptor, referred to as cC1qR because it binds the collagen-like domain of C1q. This C1qR variant was shown to be calreticulin; a 126-kDa receptor that modulates monocyte phagocytosis. gC1qR is not a membrane-bound molecule, but rather a secreted soluble protein with affinity for the globular regions of C1q, and may act as a fluid-phase regulator of complement activation.

Decay accelerating factor (DAF) (CD55) is composed of four SCRs plus a serine/threonine-enriched domain that is capable of extensive O-linked glycosylation. DAF is attached to cell membranes by a glycosyl phosphatidyl inositol (GPI) anchor and, through its ability to bind C4b and C3b, it acts by dissociating the C3 and C5 convertases. Soluble versions of DAF (sDAF) have been shown to inhibit complement activation.

C1 inhibitor, a member of the “serpin” family of serine protease inhibitors, is a heavily glycosylated plasma protein that prevents fluid-phase C1 activation. C1 inhibitor regulates the classical pathway of complement activation by blocking the active site of C1r and C1s and dissociating them from C1q.

Peptide inhibitors of complement activation include C5a and other inhibitory molecules include Fucan.

### Synapse Loss

Synapses are asymmetric communication junctions formed between two neurons, or, at the neuromuscular junction (NMJ) between a neuron and a muscle cell. Chemical synapses enable cell-to-cell communication via secretion of neurotransmitters, whereas in electrical synapses signals are transmitted through gap junctions, specialized intercellular channels that permit ionic current flow. In addition to ions, other molecules that modulate synaptic function (such as ATP and second messenger molecules) can diffuse through gap junctional pores. At the mature NMJ, pre- and postsynaptic membranes are separated by a synaptic cleft containing extracellular proteins that form the basal lamina. Synaptic vesicles are clustered at the presynaptic release site, transmitter receptors are clustered in junctional folds at the postsynaptic membrane, and glial processes surround the nerve terminal.

Synaptogenesis is a dynamic process. During development, more synapses are made than ultimately will be retained. Therefore, the elimination of excess synaptic inputs is a critical step in synaptic circuit maturation. Synapse elimination is a competitive process that involves interactions between pre- and postsynaptic partners. In the CNS, as with the NMJ, a developmental, activity-dependent remodeling of synaptic circuits takes place by a process that may involve the selective stabilization of coactive inputs and the elimination of inputs with uncorrelated activity. The anatomical refinement of synaptic circuits occurs at the level of individual axons and dendrites by a dynamic process that involves rapid elimination of synapses. As axons branch and remodel, synapses form and dismantle with synapse elimination occurring rapidly.

In addition to the normal developmental loss, synapse loss is an early pathological event common to many neurodegenerative disorders, and is the best correlate to the cognitive impairment. Studies in the brains of patients with pre-clinical Alzheimer's disease (AD), as well as in transgenic animal models have shown that synaptic damage occurs early in disease progression. This early disruption of synaptic connections in the brain results in neuronal dysfunction that, in turn, leads to the characteristic symptoms of dementia and/or motor impairment observed in several neurodegenerative disorders.

Several molecules involved in AD and other neurodegenerative disorders play an important role in synaptic function. For example, AβPP has a preferential localization at central and peripheral synaptic sites. In transgenic mice, abnormal expression of mutant forms of AβPP results not only in amyloid deposition, but also in widespread synaptic damage. This synaptic pathology occurs early and is associated with levels of soluble A131-42 rather than with plaque formation. Other neurodegenerative diseases where gene products have been shown to be closely associated with synaptic complexes include Huntington's disease (HD) and myotonic dystrophy (DM). Huntingtin (HTT) is a membrane-bound protein with a distribution very similar to that of synaptic vesicle protein synaptophysin. Studies in human brain detected HTT in perikarya of some neurons, neuropil, varicosities and as punctate staining likely to be nerve endings. The serine/threonine kinase (DMK), which is the gene product of the DM gene, has been found to localize post-synaptically at the neuromuscular junction of skeletal muscle and at intercalated discs of cardiac tissue. DMK was also found at synaptic sites in the cerebellum, hippocampus, midbrain and medulla.

Antibodies disclosed herein may be used to inhibit synapse loss. Inhibiting synapse loss results in maintenance of or reduced loss of synapses, where a decrease would otherwise occur.

### Blood Brain Barrier

As used herein, the “blood-brain barrier” (BBB) refers to the barrier between the peripheral circulation and the brain and spinal cord. The BBB is formed by tight junctions within the brain capillary endothelial plasma membrane. The formation of such tight junctions creates an extremely tight barrier that restricts the transport of molecules into the brain, even molecules as small as urea, molecular weight of 60 Da. The blood-brain barrier within the brain, the blood-spinal cord barrier within the spinal cord, and the blood-retinal barrier within the retina, are contiguous capillary barriers within the central nervous system (CNS), and are collectively referred to as the blood-brain barrier or BBB. The disclosure provides compositions and methods that include an antibody that binds to a BBB receptor mediated transport system, coupled to an agent for which transport across the BBB is desired, e.g., a neurotherapeutic agent. The compositions and methods of the disclosure may utilize any suitable structure that is capable of transport by the selected endogenous BBB receptor-mediated transport system, and that is also capable of attachment to the desired agent.

The BBB has been shown to have specific receptors that allow the transport from the blood to the brain of several macromolecules; these transporters are suitable as transporters for compositions of the disclosure. Endogenous BBB receptor-mediated transport systems useful in the disclosure include those that transport insulin, transferrin, insulin-like growth factors 1 and 2 (IGF1 and IGF2), leptin, and lipoproteins. In some embodiments, the disclosure utilizes a structure that is capable of crossing the BBB via the endogenous insulin BBB receptor-mediated transport system, e.g., the human endogenous insulin BBB receptor-mediated transport system.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the disclosure when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the antibodies of the disclosure to facilitate transport across the epithelial wall of the blood vessel. Alternatively, drug delivery behind the BBB may be pursued, e.g., by intrathecal delivery of agents directly to the cerebrospinal fluid, as through an Ommaya reservoir.

### Conditions of Interest

Representative conditions of interest include a variety of neurodegenerative diseases, autoimmune conditions, inflammatory conditions, complement-mediated eye conditions, metabolic disorders, and other conditions.

The term “neurodegenerative disease” or “neurodegenerative condition” is used in the broadest sense and includes any pathological state involving neuronal degeneration. Such diseases are characterized by the death of neurons in different regions of the nervous system and the subsequent functional deterioration of the affected parts, which include deterioration of cognitive functions and/or damage, dysfunction, or complications that may be characterized by neurological, neurodegenerative, physiological, psychological, or behavioral aberrations.

Various neurodegenerative conditions are of interest for the present methods of preventing, reducing risk of developing, or treating a neurodegenerative disorder, comprising administering an antibody that binds to complement component C4 or the C4b portion of C4. Such conditions include Alzheimer's disease, amyotrophic lateral sclerosis, multiple sclerosis, glaucoma, optic neuritis, spinal muscular atrophy, frontotemporal dementia, Parkinson's disease, Huntington's disease, schizophrenia and the like.

The antibodies of the present disclosure may also be useful in a variety of neurodegenerative conditions for the present methods of inhibiting synapse loss e.g., Alzheimer's disease, amyotrophic lateral sclerosis, multiple sclerosis, Downs Syndrome, glaucoma, optic neuritis, spinal muscular atrophy, frontotemporal dementia, Parkinson's disease, Huntington's disease, schizophrenia and the like.

Synapse loss is a significant correlate of cognitive decline that serves as a critical hallmark of neurodegenerative diseases. For example, microglia prune developing synapses and regulate synaptic plasticity and function. Disruptions in microglia-synapse interactions contribute to synapse loss and dysfunction, including cognitive impairment in neurodegenerative diseases. Furthermore, disruption of immune-related molecules or receptors expressed on microglia, such as complement proteins or complement and fractalkine receptors, results in synaptic and wiring abnormalities in both prenatal and postnatal brain development implicating microglia in sculpting synaptic connectivity.

Alzheimer's disease is a progressive, inexorable loss of cognitive function associated with an excessive number of senile plaques in the cerebral cortex and subcortical gray matter, which also contains ˜−amyloid and neurofibrillary tangles consisting of tau protein. The common form affects persons >60 yr old, and its incidence increases as age advances. It is the most common form of neurodegenerative dementia and accounts for more than 65% of the dementias in the elderly and 50-80% of all age-related dementia.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation. Alzheimer's disease is characterized by the progressive and irreversible alteration of cognitive functions, such as memory, leading inexorably to the loss of autonomy for patients with Alzheimer's disease. The pathology is linked with aging and occurs most commonly around 65 years old. Its prevalence (5% over 65 years of age and 20% after 80 years) constitutes an economic and social burden for Alzheimer's disease patients and their family.

In the course of the disease, synapses, and ultimately neurons are lost within the cerebral cortex, hippocampus, and subcortical structures (including selective cell loss in the nucleus basalis of Meynert), locus coeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease. Two of the pathological hallmarks of Alzheimer's disease (extracellular neuritic plaques, in which the amyloid β-protein (Aβ) is the principal component, and intraneuronal tangles, which are non-membrane bound masses of paired helical filaments) are composed primarily of hyperphosphorylated tau. A frequent observation in brains of both Alzheimer's disease patients and animal models of Alzheimer's disease is the surrounding of neuritic amyloid plaques by highly reactive, phagocytic microglia.

In the Alzheimer's disease brain, complement proteins are upregulated and localized to neurotic plaques. Moreover, Aβ has been shown to bind and regulate the expression and localization of complement. Genetic deletion of C1qa in an Alzheimer's disease mouse model resulted in less plaque-related neuronal damage and gliosis and thus complement proteins may have multiple roles in plaque-related pathology.

Early signs and symptoms of Alzheimer's disease include memory impairment, such as difficulty remembering events; difficulty concentrating, planning or problem-solving; problems finishing daily tasks at home or at work; confusion with location or passage of time; having visual or space difficulties, such as not understanding distance in driving, getting lost or misplacing items; language problems, such as word-finding problems or reduced vocabulary in speech or writing; using poor judgment in decisions; withdrawal from work events or social engagements; changes in mood, such as depression or other behavior and personality changes.

Diagnosis for Alzheimer's disease includes review of medical history, medication history, and symptoms, which include impaired memory or thinking, changes in personality or behaviors, and evaluation of changes in daily life. Changes in cerebrospinal fluid Aβ and tau levels are useful in diagnosis and early detection of Alzheimer's disease. The degeneration of brain cells may show up in a variety of scans, including a CT scan (computerized tomography) or MM (magnetic resonance imaging).

Amyotrophic lateral sclerosis (ALS) is a rapidly progressive, invariably fatal neurodegenerative disease that attacks motor neurons. Damaged motor neurons are able to activate microglia, astrocytes and the complement system, which further can influence each other and contribute to neurodegeneration. Motor neurons are able to activate M1 microglia and the classical complement pathway, which exerts harmful actions toward motor neurons.

Infiltrating peripheral immune cells, including complement proteins, appear to correlate with disease progression. This process is known as neuroinflammation, which includes activation of glial cells. This is known to occur in ALS and other neurodegenerative diseases. Gene expression profiling indicates that inflammatory cascades are activated prior to the initiation of the neurodegenerative process. This suggests that the immune system could already be involved in the presymptomatic phase of ALS.

Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 yr; 50% die within 3 yr of onset, 20% live 5 yr, and 10% live 10 yr. Diagnostic features include onset during middle or late adult life and progressive, generalized motor involvement without sensory abnormalities. Nerve conduction velocities are normal until late in the disease.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Preventing synapse loss may maintain neuron function in these patients.

Amyotrophic lateral sclerosis (ALS) is difficult to diagnose early because it may appear similar to several other neurological diseases. Tests to rule out other conditions may include: Electromyogram (EMG), Nerve conduction study (NCV), Magnetic resonance imaging (MRI), Blood and urine tests, Spinal tap (lumbar puncture), and Muscle biopsy. Certain EMG findings can support the diagnosis of ALS. Specific abnormalities in the NCV results may suggest, for example, that the subject has a form of peripheral neuropathy (damage to peripheral nerves) or myopathy (muscle disease) rather than ALS. While MRI scans are often normal in subjects with ALS, they can reveal evidence of other problems that may be causing the symptoms, such as a spinal cord tumor, a herniated disk in the neck, syringomyelia, or cervical spondylosis.

Down Syndrome is a chromosomal disorder usually resulting in mental retardation, a characteristic facies, and many other typical features, including microcephaly and short stature. In about 95% of cases, there is an extra whole chromosome 21. At autopsy, adult Down syndrome brains show the typical microscopic findings of Alzheimer's disease, and many persons also develop the associated clinical signs.

Parkinson's Disease is the second most frequent neurodegenerative disorder. It is known as an idiopathic, slowly progressive, degenerative CNS disorder characterized by slow and decreased movement, muscular rigidity, resting tremor, and postural instability. In primary Parkinson's disease, the pigmented neurons of the substantia nigra, locus coeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

Early symptoms of Parkinson's disease are subtle and occur gradually. For example, affected subjects may sense mild tremors or have difficulty getting out of a chair. They may notice that they speak too softly or that their handwriting is slow and looks cramped or small. This very early period may last a long time before the more classic and obvious symptoms appear.

As the disease progresses, symptoms may begin to interfere with daily activities. For example, shaking or tremor may make it difficult to hold utensils steady or read a newspaper. Tremor is usually the symptom that causes people to seek medical help.

Subjects with Parkinson's often develop a so-called parkinsonian gait that includes a tendency to lean forward, small quick steps as if hurrying forward (called festination), and reduced swinging of the arms. They also may have trouble initiating or continuing movement, which is known as freezing. Symptoms often begin on one side of the body or even in one limb on one side of the body. As the disease progresses, it eventually affects both sides. However, the symptoms may still be more severe on one side than on the other.

The four primary symptoms of Parkinson's are tremor, rigidity, slowness of movement (bradykinesia), and impaired balance (postural instability). Tremor often begins in a hand, although sometimes a foot or the jaw is affected first. It is most obvious when the hand is at rest or when a subject is under stress. It usually disappears during sleep or improves with a deliberate movement. Rigidity, or a resistance to movement, is a major sign of Parkinson's. It becomes obvious when another subject tries to move the individual's arm, such as during a neurological examination. The arm will move only in ratchet-like or short, jerky movements known as “cogwheel” rigidity. Bradykinesia, or the slowing down and loss of spontaneous and automatic movement, is particularly frustrating because it may make simple tasks somewhat difficult. Activities once performed quickly and easily, such as washing or dressing, may take several hours. Postural instability, or impaired balance, causes subjects with Parkinson's to fall easily. Subjects also may develop a stooped posture with a bowed head and droopy shoulders.

Diagnosis is based on a subject's medical history and a neurological examination, but the disease can be difficult to diagnose accurately. Early signs and symptoms of Parkinson's may sometimes be dismissed as the effects of normal aging. A doctor may need to observe the person for some time until it is clear that the symptoms are consistently present. Improvement after initiating medication is another important hallmark of Parkinson's disease. Sometimes brain scans or laboratory tests can rule out other diseases. However, computed tomography (CT) and magnetic resonance imaging (MRI) brain scans of people with Parkinson's usually appear normal. Recently, the FDA (Food and Drug Administration) has approved an imaging technique known as DaTscan, which may help to increase accuracy of the diagnosis of Parkinson's disease.

Huntington's disease (HD) is a hereditary progressive neurodegenerative disorder characterized by a progressive course of disease until death, and the development of emotional, behavioral, and psychiatric abnormalities; loss of previously acquired intellectual or cognitive functioning; and movement abnormalities (motor disturbances). The classic signs of HD include the development of chorea—or involuntary, rapid, irregular, jerky movements that may affect the face, arms, legs, or trunk—as well as the gradual loss of thought processing and acquired intellectual abilities (dementia). There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

HD may be caused by a mutation with expanded CAG repeats in the huntingtin (htt) protein. Mutant htt (mhtt) in the striatum is assumed to be one of the main reasons for neurodegeneration.

Knowledge about pathophysiology has rapidly improved suggesting influences of excitotoxicity, mitochondrial damage, free radicals, and inflammatory mechanisms. Both innate and adaptive immune systems may play an important role in HD. Activation of microglia with expression of proinflammatory cytokines, impaired migration of macrophages, and deposition of complement factors in the striatum indicate an activation of the innate immune system. In HD, striatal and cortical neurodegeneration may be triggered by inflammation. Conversely, increased inflammation in HD might be the response to neuronal death induced by mhtt toxicity.

Microglia are the primary mediators of neuroinflammation and the likely key players in the pathogenesis of neurodegenerative diseases. In HD, the main neuropathological changes take place in the nuclei of the basal ganglia and are characterized by degeneration and neuronal loss. Consequently, and in line with the neurodegenerative process, astrocytes and microglia cells increase in number in the affected regions. Under physiological conditions, microglia are in a resting state and contribute to innate immune responses by producing anti-inflammatory and neurotrophic factors. In brains of mhtt carriers, microglia are activated even before onset of symptoms. Based on ferritin accumulation and Iba1 immunostaining, increased microglial activation was also shown in the R6/2 mouse model.

In HD, the complement system may be activated by peptides such as mhtt. Activation of the complement systems triggers a cascade of processes including cytokine release, enhancing phagocytosis of antigens, attracting macrophages and neutrophils, cell lysis, and, amongst others, production of anaphylatoxins with its central component C3. In HD, several complement factors, notably C1q, C4, and C3, are expressed in the striatum comprising neurons, astrocytes, and myelin.

Symptoms of HD may include, but are not limited to: slight, uncontrollable muscular movements, stumbling and clumsiness, lack of concentration, lapses of short-term memory, depression, and changes of mood, sometimes including aggressive or antisocial behavior.

The rate of progression of Huntington's disease varies, but generally, it develops over 15-25 years. Later in the illness, people may experience different symptoms, which include: involuntary movements, difficulty in speech and swallowing, weight loss; emotional changes include stubbornness, frustration, mood swings, and depression.

A diagnosis of Huntington's disease is suspected based on the appearance of specific symptoms such as those described above. Some of the steps taken to diagnose Huntington's disease include the following: (1) a detailed physical examination to assess whether disease onset has started. The test will analyze signs of involuntary body movements. Unintentional, random and abrupt movements will often lead to a diagnosis of Huntington's being suspected; (2) a detailed psychological examination may also be performed to check for signs of decline in motor, behavioral and cognitive function; (3) medical imaging techniques, such as computerized tomography (CT) and magnetic resonance imaging (MM) may reveal atrophy of the caudate nuclei, which is observed in the early stages of Huntington's disease; (4) functional neuroimaging techniques such as fMRI and PET (Positron emission tomography) may reveal changes in brain activity before physical symptoms start to develop; (5) genetic testing can be performed to check whether a subject is at risk of Huntington's disease. A blood sample is taken and checked for the mutation in the patient's two copies of the Huntingtin (HTT) gene which codes for the huntingtin protein. The mutation that is checked for is an expansion mutation of the cytosine-adenine-guanine (CAG) triplet found in the HTT gene on chromosome 4. The mutated gene codes for a huntingtin protein that is abnormal and gradually damages brain cells. A subject with the faulty gene will develop the disease but the age at which this will happen cannot be determined.

Several other diseases may have a similar symptom profile to Huntington's disease, such as neuroferritinopathy, chorea acanthocytosis and X-linked McLeod syndrome.

Multiple Sclerosis (MS) belongs to a larger group of inflammatory demyelinating diseases of the CNS, which include, besides the different manifestations of MS, acute disseminated leukoencephalitis, acute optic neuritis, Devic's neuromyelitis optica and Balo's concentric sclerosis. Although these diseases differ in clinical course, imaging pathology and immunopathogenesis, they share some essential structural features of their lesions, as they all occur on a background of inflammatory reaction composed of lymphocytes, activated macrophages and microglia and show demyelination. MS is a chronic inflammatory and neurodegenerative disease affecting over 2.5 million individuals worldwide. The pathological hallmark of MS is white matter demyelination but there are other features such as axonal and neuronal damage, grey matter demyelination, composition of vascular cuffs (monocytes, T cells, B cells, plasma cells), loss of oligodendrocytes and anatomical lesions that vary between patients.

MS may also be characterized by various symptoms and signs of CNS dysfunction, with remissions and recurring exacerbations. The complement system is implicated in the pathogenesis of MS and is associated with demyelinating pathology in multiple sclerosis (MS) lesions, where macrophages predominate among infiltrating myeloid cells. Multiple sclerosis lesions have been classified on the basis of pathological patterns where pattern II lesions are defined by presence of antibodies and activated complement product deposition. The most common presenting symptoms are paresthesia in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for some patients, the course can be rapidly progressive.

The clinical course of MS is characterized by relapses and/or disease progression. Relapses are defined as newly appearing neurological symptoms in the absence of fever or infections that last for more than 24 h. Relapses may fully recover over days or weeks, or lead to persistent residual deficits. Disease progression is a steady worsening of symptoms and signs over at least 6 months. Typical clinical presentations of relapses are optic neuritis (in about 20% of cases this is the initial symptom), sensory deficits or cerebellar dysfunction, whereas progressive courses are often characterized by spinal symptoms such as gait ataxia, paresis and spasticity. Disability is commonly measured using the Expanded Disability Status Scale (EDSS).

In 85% of patients, a relapse is the initial clinical event leading to relapsing-remitting MS (RRMS). After a median time of approximately 19 years (at a mean age of 40 years), RRMS changes to secondary progressive MS (SPMS) in 75% of patients. In SPMS, a steady worsening of symptoms dominates the clinical course with superimposed relapses in 40% of patients, especially early after conversion.

In 15% of patients, the disease is progressive from the onset (primary progressive MS, PPMS), with a mean age of onset of 40 years. Amongst these patients, 40% experience superimposed relapses (progressive-relapsing MS). Clinical symptoms are dominated by dysfunctions of the corticospinal tracts and disease courses are more severe compared to initially relapsing forms of MS. For example, an EDSS score of 6, i.e. the need for unilateral support to walk at least 100 m, is reached in 7 years in PPMS compared to 12.5 years in SPMS patients. However, the disease course is not predictable for the individual subject.

The diagnosis of MS is based on the demonstration of MS-typical CNS lesions disseminated in space (DIS) and time (DIT) based upon clinical findings alone or a combination of clinical and MRI findings. In 2010, the so-called McDonald criteria, first published in 2001, were revised for the second time by the International Panel on Diagnosis of MS based on new evidence and consensus to facilitate earlier diagnosis of MS and to increase the sensitivity and specificity of diagnosis. DIS lesions detected using MRI can now be used for diagnosis, with at least one T2 lesion in two out of four CNS regions considered typical of MS. For DIS, symptomatic lesions in patients with brainstem and/or spinal cord symptoms are excluded. DIT lesions detected using MM can be demonstrated by the presence of a new T2 and/or gadolinium (Gd)-enhancing lesion(s) on follow-up Mill, with reference to a baseline scan irrespective of the timing of the baseline MRI, or by the simultaneous presence of asymptomatic Gd-enhancing and non-enhancing lesions at any time. Therefore, at earliest, RRMS can be diagnosed after a single relapse with a single MM showing asymptomatic Gd-enhancing and non-enhancing lesions that are disseminated in space.

For the diagnosis of PPMS, 1 year of disease progression and several additional criteria are required, such as the presence of lesions in the brain, DIS in the spinal cord, and positive cerebrospinal fluid.

In general, at least one clinical attack must be corroborated by findings on neurological examination, visual evoked potentials or MRI consistent with the clinical presentation. Furthermore, the exclusion of alternative diagnoses is mandatory when applying the criteria. Besides the correct interpretation of the clinical symptoms and signs as well as the MRI, additional assessments such as lumbar puncture, blood tests and visual evoked potentials are useful for excluding other diseases and to support the diagnosis of MS.

Glaucoma is a common neurodegenerative disease that affects retinal ganglion cells (RGCs). Substantial effort is being expended to determine how RGCs die in glaucoma. Evidence supports the existence of compartmentalized degeneration programs in synapses and dendrites, including RGCs. Recent data, from in vitro studies and from an inherited mouse model of glaucoma, suggest that molecularly distinct degenerative pathways underlie the destruction of RGC somata and RGC axons. In various neurodegenerative diseases, axons, dendrites and synapses often degenerate well before the cells die, and there is increasing evidence that this is important for the production of clinical symptoms and signs.

Myotonic dystrophy (DM) is an autosomal dominant multisystem disorder characterized by dystrophic muscle weakness and myotonia. The molecular defect is an unstable and expanded trinucleotide (CTG) repeat in the 3′ untranslated region of the myotonin-protein kinase gene on chromosome 19q. Also, neurofibrillary tangles (NFT) are observed in patient brains, which distinguishes DM as a tauopathy. Tauopathies are a group of nearly 30 neurodegenerative diseases that are characterized by intraneuronal protein aggregates of the microtubule-associated protein Tau (MAPT) in patient brains. Furthermore, a number of neurodegenerative diseases involve the dysregulation of splicing regulating factors and have been characterized as spliceopathies. Thus, myotonic dystrophies are pathologies resulting from the interplay among spliceopathy, and tauopathy. Symptoms can occur at any age, and the range of clinical severity is broad. Myotonia is prominent in the hand muscles, and ptosis is common even in mild cases. In severe cases, marked peripheral muscular weakness occurs, often with cataracts, premature balding, hatchet facies, cardiac arrhythmias, testicular atrophy, and endocrine abnormalities (e.g., diabetes mellitus). Mental retardation is common. Severely affected persons die by their early 50s.

Schizophrenia is a debilitating and common mental disorder that impairs mental and social functioning and often leads to the development of comorbid diseases. A family history of schizophrenia is the most significant risk factor, even though schizophrenia appears to be a polygenic disorder with environmental and developmental factors mediating a person's likelihood of becoming schizophrenic. While involvement of genetic factors in the etiopathogenesis of schizophrenia is clear, no specific causal gene, such as huntingtin for Huntington's disease, has been reported for this mental disorder. Recent studies however indicate that the risk for developing schizophrenia is increased in subjects expressing higher levels of a specific version of C4 called C4A. These findings are supported by mouse model studies, where knockdown of C4 led to reductions in synaptic pruning and supports the hypothesis that increased C4A expression and levels led to increased risk of developing schizophrenia.

Schizophrenia is associated with disturbance of neuronal connectivity. It is known that initial and major risks for the disease occur during neurodevelopment, although onset of the disease occurs in juveniles and young adulthood. Autopsied brains from subjects with the disease suggested that synapse loss may be associated with the pathology of schizophrenia. The pathology appears to include dysfunction of glutamatergic neurotransmission in the prefrontal cortex of schizophrenia subjects. It is unknown if the range of severity and clinical manifestations reflect problems in different brain regions, in different causalities, or in different diseases that share some phenotypic features.

Schizophrenia is characterized by positive and negative symptoms that can influence a subject's thoughts, perceptions, speech, affect, and behaviors. Positive symptoms include hallucinations, voices that converse with or about the patient, and delusions that are often paranoid. Negative symptoms include flattened affect, loss of a sense of pleasure, loss of will or drive, and social withdrawal.

Schizophrenia is also characterized by disorganized thought, which is manifested in speech and behavior. Disorganized speech may range from loose associations and moving quickly through multiple topics to speech that is so muddled that it resembles schizophasia (commonly referred to as “word salad”). Schizophasia is speech that is confused and repetitive, and that uses words that have no apparent meaning or relationship to one another. Disorganized behavior may lead to difficulties in performing daily living activities, such as preparing a meal or maintaining hygiene. It also can manifest as childlike silliness or outbursts of unpredictable agitation.

No single sign or symptom is pathognomonic of schizophrenia. To make a definitive diagnosis, signs and symptoms must be present for a significant portion of one month (or a shorter period if successfully treated), and some must be present for at least six months. These symptoms also must be associated with marked social and occupational dysfunction.

There are five types of schizophrenia: paranoid, disorganized, catatonic, undifferentiated, and residual. Paranoid type is characterized by a preoccupation with one or more delusions or frequent auditory hallucinations; cognitive function and affect remain relatively well preserved. Disorganized type is characterized by disorganized speech and behavior, as well as flat or inappropriate affect. Catatonic type has at least two of the following features: immobility (as evidenced by stupor or catalepsy); excessive, purposeless motor activity; extreme negativism (e.g., resistance to all instructions, maintenance of rigid posture, mutism); or peculiarities of voluntary movement (e.g., posturing, prominent mannerisms, grimacing). A subject is said to have undifferentiated schizophrenia if none of the criteria for paranoid, disorganized, or catatonic types are met. Residual type is characterized by the continued presence of negative symptoms (e.g., flat affects, poverty of speech) and at least two attenuated positive symptoms (e.g., eccentric behavior, mildly disorganized speech, odd beliefs). A patient is diagnosed with residual type if he or she has no significant positive psychotic features. Of note, this classic typing of schizophrenia can be limiting because subjects often are difficult to classify. For that reason, an alternative three-factor dimensional model is given. The three factors are psychotic, disorganized, and negative (deficit). The symptoms are categorized as absent, mild, moderate, or severe.

The onset of schizophrenia can be abrupt or insidious. Subjects typically undergo a prodromal phase marked by a slow and gradual development of symptoms, such as social withdrawal, loss of interest in school or work, deterioration in hygiene and grooming, unusual behavior, or outbursts of anger. Family members can find this behavior disturbing and difficult to interpret. They may assume that the person is just “going through a phase.” Eventually, the appearance of active-phase symptoms (e.g., psychosis) marks the disturbance as schizophrenia.

Other symptoms that may suggest schizophrenia, and may be used in a differential diagnosis of schizophrenia, including the following diagnoses and distinguishing features: brief psychotic disorder (e.g., presence of delusions, hallucinations, disorganized speech, or grossly disorganized or catatonic behavior lasting at least one day but less than one month); delirium (multiple underlying etiologies; symptoms often similar to positive symptoms of schizophrenia but with a much shorter course); delusional disorder (e.g., delusions are not bizarre, and there are no other characteristics of schizophrenia); medical illnesses (e.g., illnesses that may cause schizophrenia-like symptoms include hepatic encephalopathy, hypoglycemia, electrolyte abnormalities (for example, hyponatremia, hypercalcemia, hypocalcemia, hypomagnesemia), and sepsis); medication-induced disorder (for example, medications that may cause schizophrenia-like symptoms include anticholinergics, anxiolytics, digoxin, phenytoin (Dilantin), steroids, narcotics, and cimetidine (Tagamet)); mood disorders with psychotic features (for example, no major depressive, manic, or mixed episodes have occurred concurrently with active phase symptoms; or, if they have occurred, their total duration has been brief relative to the duration of the active and residual symptoms); pervasive developmental disorder (i.e., recognized during infancy or early childhood; absence of delusions and hallucinations); psychotic disorder NOS (diagnosis is made if there is insufficient information available to choose between schizophrenia and other psychotic disorders); schizophreniform disorder (lasts one to six months; diagnosis does not require a decline in functioning); schizotypal personality disorder (pervasive patterns of social and interpersonal deficits beginning in early adulthood; accompanied by eccentric behavior and cognitive or perceptual distortions), or substance abuse (multiple substances (e.g., hallucinogens, narcotics, alcohol) or substance withdrawal, as withdrawal from these substances can cause delusions and hallucinations.

Despite the stability of the diagnostic criteria for schizophrenia, diagnosis often changes over time. For example, delirium can have features that are similar to the active symptoms of schizophrenia (e.g., hallucinations, delusions). However, one of the differences between schizophrenia and delirium is the timing; signs and symptoms of schizophrenia generally develop over weeks to months, whereas delirium usually has a much more rapid onset. Because many medical illnesses can cause delirium, the diagnosis of new-onset schizophrenia is often made cautiously in subjects who have an existing serious medical illness.

There also are racial disparities in the diagnosis of schizophrenia. For example, black persons are more likely than other racial groups to have symptoms attributed to schizophrenia, and Hispanics are more likely to be diagnosed with major depression when presenting with psychotic symptoms. A complete history chronicling the development of signs and symptoms is crucial when diagnosing schizophrenia.

Optic Neuritis is an inflammation of the optic nerve, the bundle of nerve fibers that transmits visual information from the eye to the brain. It is also known as optic papillitis (when the head of the optic nerve is involved) and retrobulbar neuritis (when the posterior of the nerve is involved). Pain and temporary vision loss are common symptoms of optic neuritis. Optic neuritis is highly associated with multiple sclerosis, a condition mentioned earlier that causes inflammation and damage to nerves in your brain and spinal cord. In some subjects, signs and symptoms of optic neuritis may be the first indication of multiple sclerosis.

Major symptoms of optic neuritis are sudden loss of vision (partial or complete), sudden blurred or “foggy” vision, and pain on movement of the affected eye. Early on, symptoms characteristic of Multiple Sclerosis are evident (twitching, no coordination, slurred speech, frequent episodes of partial vision loss or blurred vision). Episodes of “disturbed/blackened” rather than blurry indicate moderate stage and require immediate medical attention to prevent further loss of vision. Other early symptoms are reduced night vision, light sensitivity and regular blood shot eyes. Subjects with optic neuritis may lose some of their color vision in the affected eye (especially red), with colors appearing subtly washed out compared to the other eye. Subjects may also experience difficulties judging movement in depth, which can be particular troublesome during driving (known as the Pulfrich effect). Likewise, transient worsening of vision with increase of body temperature (“Uhthoff s phenomenon”) and glare disability is a sign of optic neuritis.

Typical optic neuritis develops over a 7- to 10-day period and begins to resolve within 2-3 weeks. Numerous studies have found evidence of persistent retinal thinning, optic nerve atrophy, and reduced amplitude and increased latency of visual-evoked potentials (VEPs), consistent with chronic demyelination and neuroaxonal loss as sequelae of acute optic neuritis. Although patients with optic neuritis frequently regain visual acuity to a large extent as measured by full-contrast letter charts (e.g., Snellen charts), low-contrast acuity/sensitivity reveals permanent deficits and is a better predictor of impairment for daily activities that require vision, such as reading, facial recognition, and driving. Persistent deficits in low-contrast letter acuity characteristic of optic neuritis are better measured using Sloan charts (Sloan low-contrast letter acuity [SLCLA]) that include versions with 2.5% and 1.25% contrast levels to better stratify deficits. The pattern of visual field defects may help distinguish optic neuritis from other neuropathies. In optic neuritis, a central scotoma is common and Humphrey central visual field perimetry frequently shows diffuse loss, whereas peripheral, latitudinal, or other defects may occasionally be evident on formal perimetry. Color vision is commonly affected in optic neuritis, but there is no consistent pattern of dyschromatopsia. More complex visual functions, such as motion perception, are also frequently affected by optic neuritis. Binocular summation (improved vision with binocular viewing) has also been shown to be reduced, and in some instances patients demonstrate binocular inhibition (worse vision with binocular viewing), perhaps reflecting concomitant disease activity in postgeniculate pathways in some subjects. Thus, evaluation of visual function after optic neuritis requires multiple tests to ensure comprehensive assessment of the potential deficits. Standard VEPs elicited by visual stimuli and measured in the occipital cortex can be used to detect functional changes in the visual pathway, including the optic nerve. In multifocal VEPs (mfVEPs), visual stimuli are provided independently to localized regions of a wider visual field (48°) and responses to the stimuli are measured individually, allowing for a more detailed analysis of visual function covering a much larger area of the visual pathway than standard VEPs. The severity of optic neuritis and the extent of inflammation are correlated with an acute reduction in the amplitude of VEPs.

Spinal muscular atrophy (SMA) is an autosomal-recessive disorder characterized by degeneration of motor neurons in the spinal cord and caused by mutations in the survival motor neuron 1 gene, SMN1. The severity of SMA is variable and inversely correlated with the amount of SMN protein produced from a second gene called SMN2. The SMN2 gene produces ˜10% of the SMN messenger RNA (mRNA) transcript compared to the SMN1 gene.

The predominant features of SMA are muscle weakness and atrophy. Weakness is usually symmetric, with proximal muscles more affected than distal groups as in NP7. Over the last 125 years, reports detailing the clinical manifestations and wide range of clinical severity have all recognized and emphasized the seminal pathology as anterior horn cell degeneration, as well as the pertinent clinical features of symmetric, proximal predominant extremity weakness that also affects axial, intercostal, and bulbar musculature. The multiple described phenotypes were eventually formalized into a classification scheme at an International Consortium on Spinal Muscular Atrophy sponsored by the Muscular Dystrophy Association in 1991. This classification highlighted 3 SMA types based on the highest level of motor function (i.e., sitting or standing) and age of onset. Subsequent modifications divided the type 3 category by age of onset, added a type 4 for adult-onset cases, and included a type 0 for patients with prenatal onset and death within weeks. Although there are degrees of severity even within an individual type, and as many as 25% of patients elude precise classification, this scheme remains relevant in the genetic era and provides useful clinical and prognostic information.

As mentioned earlier, subjects with SMA lack a functioning SMN1 gene and are thus dependent on their SMN2 gene, however inefficient, to produce the SMN protein necessary for survival. Thus, SMA is caused by a deficiency of the SMN protein that results in selective motor neuron loss. SMN is found throughout the cytoplasm and nucleus where it functions as part of a multiprotein complex, the SMN complex, which plays an essential role in spliceosomal small nuclear ribonuclear protein biogenesis and pre-mRNA splicing. Small nuclear ribonuclear protein biogenesis is altered in the cells of SMA mice. The SMN protein has also been detected in the axons of motor neurons.

Gastrointestinal complications are common in individuals with SMA, and it is not clear if this is owing to immobility and nutritional deficiencies or whether there is a primary defect is gastrointestinal mobility. Infants with type 1 SMA often have prolonged feeding times and tire quickly. This reduction in feeding can be the first sign of progressive weakness and can lead to failure to thrive and aspiration. Other associated problems include gastrointestinal reflux, delayed gastric emptying, and constipation. These complications are also seen in subjects who, for other reasons, cannot sit or stand, and are less commonly seen in ambulant individuals with SMA. Malnutrition, secondary to decreased oral intake, can also be an insidious problem for some type 2 SMA children and adolescents.

Weakness and impaired mobility predispose to numerous musculoskeletal issues for SMA subjects. Early recognition and appropriate management are helpful in maintaining function, preventing deterioration in vital capacity, and improving quality of life. Scoliosis occurs in almost all non-ambulant individuals with SMA. When untreated, scoliosis causes chest cage deformities with subsequent respiratory restriction.

Frontotemporal dementia (FTD) is a neurodegenerative disorder characterized by progressive deficits in either behavior and personality changes or language disturbance. FTD is an umbrella term for a broad spectrum of diseases including Progressive Supranuclear Palsy, Corticobasal Degeneration, and Amyotrophic Lateral Sclerosis. FTD is often misdiagnosed in the early stage, either as a psychiatric disorder, or as a different type of dementia such as Alzheimer's disease (AD). Because of the close similarity of behavioral changes in patients with frontotemporal dementia to those seen in patients with psychiatric disorders, diagnosis is challenging. Various underlying neuropathological entities lead to the FTD phenotype, all of which are characterized by selective degradation of the frontal and temporal cortices.

FTD is used for the description of a group of early onset dementias, which is the second most common dementing disorder among people under 65 years of age. However, in 25% of the cases FTD presents in old age. The estimated prevalence of FTD is 15-22/100,000 and population studies indicate an equal gender distribution. WHO estimates that dementia rates will double every 20 years, reaching 135.5 million in 2050.

Clinically FTD patients can present with one of three canonical clinical syndromes: behavioral variant FTD (bvFTD), and two language variants, semantic dementia and progression non-fluent aphasia (PNFA). bvFTD is associated with early behavioral and executive deficits, which refers to the higher-level cognitive skills that control and coordinate other cognitive abilities and behaviors. Semantic dementia, also called semantic-variant primary progressive aphasia, is a progressive disorder of semantic knowledge and naming. PNFA is characterized by progressive deficits in speech, grammar, and word aphasia. As FTD progresses, the symptoms of the three clinical variants can converge, as an initially focal degeneration becomes more diffuse and spreads to affect large regions in the frontal and temporal lobes. Over time, patients develop global cognitive impairment and motor deficits, including Parkinsonism, and motor neuron disease in some patients. Patents with end-stage disease have difficulty eating, moving, and swallowing. Death usually happens about 8 years after symptom onset and is typically caused by pneumonia or other secondary infections.

FTD can overlap with motor neuron disease/amyotrophic lateral sclerosis (MND/ALS)(FTD-MND), corticobasal syndrome (CBS) and progressive supranuclear palsy (PSP) syndrome. FTD is also a highly heritable disorder with approximately 30-50% of cases reporting positive family history, although an autosomal dominant history accounts for approximately 10% of the cases. Mutations in three genes, microtubule-associated protein tau (MAP7), progranulin (GRN) and chromosome 9 open reading frame 72 (C9orf72) genes are considered responsible for most of the familial cases, and about 10-20% of all cases with FTD.

No approved disease-modifying drugs are available for the treatment of FTD. Treatment is focused on management of behavioral symptoms. Severity of compulsion, agitation, aggressiveness, impulsivity, and aberrant eating behavior can improve with the use of selective serotonin reuptake inhibitors. Behavioral abnormalities can be managed with low doses of atypical antipsychotics. Cholinesterase inhibitors are not beneficial and can worsen behavioral abnormalities seen in patients with FTD. Memantine does not improve or delay progression of FTD symptoms.

Other conditions of interest are autoimmune conditions, inflammatory conditions, complement-mediated eye conditions or metabolic disorders, such as diabetes, obesity, rheumatoid arthritis (RA), acute respiratory distress syndrome (ARDS), remote tissue injury after ischemia and reperfusion, complement activation during cardiopulmonary bypass surgery, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, dry AMD, sarcoidosis, temporal arteritis, polyarteritis nodosa, multiple sclerosis, allo-transplantation, hyperacute rejection, hemodialysis, chronic occlusive pulmonary distress syndrome (COPD), asthma, aspiration pneumonia, Guillain-Barre syndrome, Myasthenia Gravis, Bullous Pemphigoid, or myositis.

The term “autoimmune condition” is used in the broadest sense and includes myasthenia gravis, Diabetes mellitus type 1, Hashimoto's thyroiditis, Addison's disease, Coeliac disease, Crohn's disease, pernicious anemia, Pemphigus vulgaris, vitiligo, autoimmune hemolytic anemia, paraneoplastic syndromes, a vasculitis disease, hypocomplementemic urticarial vasculitis (HUV), polymyalgia rheumatica, temporal arteritis, Wegener's granulomatosis, multiple sclerosis, Guillain-Barre syndrome, Myasthenia Gravis, Bullous Pemphigoid, or myositis.

The term “complement-associated eye condition” is used in the broadest sense and includes all eye condition pathology that involves complement, including the classical and the alternative pathways, and in particular the alternative pathway of complement. Complement-associated eye conditions include, without limitation, Chronic open-angle glaucoma, acute closed angle glaucoma, macular degenerative diseases, such as all stages of age-related macular degeneration (AMD), including dry and wet (non-exudative and exudative) forms, choroidal neovascularization (CNV), uveitis, diabetic and other ischemia-related retinopathies, and other intraocular neovascular diseases, such as diabetic macular edema, pathological myopia, von Hippel-Lindau disease, histoplasmosis of the eye, Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, dry AMD, sarcoidosis, temporal arteritis, polyarteritis nodosa, and endophthalmitis. Such conditions benefit from administration of inhibitors of complement, including inhibitors of C4, which allow maintenance, or reduced loss, of synapses. In some instances, where there has been neuronal loss, it may be desirable to enhance neurogenesis as well, e.g. through administration of agents or regimens that increase neurogenesis, transplantation of neuronal progenitors, etc. Agents that enhance synaptogenesis, such as thrombospondins, may also be administered.

Age-related Macular Degeneration (AMD) is the leading cause of blindness in the elderly worldwide. AMD is characterized by a progressive loss of central vision attributable to degenerative and neovascular changes in the macula, a highly specialized region of the ocular retina responsible for fine visual acuity. Recent estimates indicate that 14 million persons are blind or severely visually impaired because of AMD. The disease has a tremendous impact on the physical and mental health of the geriatric population and their families and is becoming a major public health burden. New discoveries, however, are beginning to provide a clearer picture of the relevant cellular events, genetic factors, and biochemical processes associated with early AMD and how AMD is a complement-associated eye condition. Two types of AMD exist, non-exudative (dry) and exudative (wet) AMD. The dry, or nonexudative, form involves atrophic and hypertrophic changes in the retinal pigment epithelium (RPE) underlying the central retina (macula) as well as deposits (drusen) on the RPE. Patients with nonexudative AMD can progress to the wet, or exudative, form of AMD, in which abnormal blood vessels called choroidal neovascular membranes (CNVMs) develop under the retina, leak fluid and blood, and ultimately cause a blinding disciform scar in and under the retina. Nonexudative AMD, which is usually a precursor of exudative AMD, is more common. The presentation of nonexudative AMD varies; hard drusen, soft drusen, RPE geographic atrophy, and pigment clumping can be present. Complement components are deposited on the RPE early in AMD and are major constituents of drusen.

### Methods of Treatment

By administering agents that inhibit complement activation, synapses can be maintained that would otherwise be lost. Such agents include an anti-C4 or anti-C4b antibody inhibitor, agents that upregulate expression of native complement inhibitors, agents that down-regulate C4, or C4b synthesis in neurons, astrocytes, microglia, endothelial, or oligodendroglial cells, agents that block complement activation, agents that block the signal for complement activation, and the like.

The methods promote improved maintenance of neuronal function in conditions associated with synapse loss. The maintenance of neural connections provides for functional improvement in neurodegenerative disease relative to untreated patients. The prevention of synapse loss may comprise at least a measurable improvement relative to a control lacking such treatment over the period of 1, 2, 3, 4, 5, 6 days or at least one week, for example at least a 10% improvement in the number of synapses, at least a 20% improvement, at least a 50% improvement, or more.

The agents of the present disclosure may be administered at a dosage that decreases synapse loss while minimizing any side-effects. It is contemplated that compositions may be obtained and used under the guidance of a physician for in vivo use. The dosage of the therapeutic formulation may vary widely, depending upon the nature of the disease, the frequency of administration, the manner of administration, the clearance of the agent from the host, and the like.

The effective amount of a therapeutic composition given to a particular patient may depend on a variety of factors, several of which may be different from patient to patient. Utilizing ordinary skill, the competent clinician will be able to tailor the dosage of a particular therapeutic or imaging composition in the course of routine clinical trials.

Therapeutic agents, e.g., inhibitors of complement, activators of gene expression, etc. can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intratracheal, etc., administration. The active agent may be systemic after administration or may be localized by the use of regional administration, intramural administration, or use of an implant that acts to retain the active dose at the site of implantation.

### Combination Treatments

The complement inhibitors of the present disclosure may be used, without limitation, conjointly with any additional treatment, such as immunosuppressive therapies, for any disease disclosed herein, neurodegenerative diseases, complement-associated eye conditions, inflammatory and autoimmune diseases.

In some embodiments, the antibodies of this disclosure may be administered in combination with an inhibitor of the alternative pathway of complement activation. Such inhibitors may include, without limitation, factor B blocking antibodies, factor D blocking antibodies, soluble, membrane-bound, tagged or fusion-protein forms of CD59, DAF, CR1, CR2, Crry or Compstatin-like peptides that block the cleavage of C3, non-peptide C3aR antagonists such as SB 290157, Cobra venom factor or non-specific complement inhibitors such as nafamostat mesilate (FUTHAN; FUT-175), aprotinin, K-76 monocarboxylic acid (MX-1) and heparin (see, e.g., T. E. Mollnes & M. Kirschfink, Molecular Immunology 43 (2006) 107-121). In some embodiments, the antibodies of this disclosure are administered in combination with an inhibitor of the interaction between the autoantibody and its autoantigen. Such inhibitors may include purified soluble forms of the autoantigen, or antigen mimetics such as peptide or RNA-derived mimotopes, including mimotopes of the AQP4 antigen. Alternatively, such inhibitors may include blocking agents that recognize the autoantigen and prevent binding of the autoantibody without triggering the classical complement pathway. Such blocking agents may include, e.g., autoantigen-binding RNA aptamers or antibodies lacking functional C4 or C4b binding sites in their Fc domains (e.g., Fab fragments or antibodies otherwise engineered not to bind C4 or C4b).

The methods of the present disclosure can find use in combination with cell or tissue transplantation to the central nervous system, where such grafts include neural progenitors such as those found in fetal tissues, neural stem cells, embryonic stem cells or other cells and tissues contemplated for neural repair or augmentation. Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. Accordingly, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of preventing, reducing risk of developing, or treating a neurodegenerative disorder, comprising administering an antibody that binds to complement component C4 or the C4b portion of C4.

2-11. (canceled)

12. The method of claim 1, wherein the antibody blocks cleavage of C4 by C1s.

13. The method of claim 1, wherein the antibody blocks cleavage of C4 by MASP-1.

14. The method of claim 1, wherein the antibody blocks cleavage of C4 by MASP-2.

15. The method of claim 1, wherein the antibody blocks cleavage of C4 by MASP-1 and MASP-2.

16. The method of claim 1, wherein the antibody blocks C4 binding to C2 or the C2a portion of C2.

17. The method of claim 1, wherein the antibody blocks C4b binding to C2a.

18. The method of claim 1, wherein the antibody blocks cleavage of C3.

19. The method of claim 1, wherein the antibody blocks C4b2a binding to C3b.

20. The method of claim 1, wherein the antibody blocks C5 binding to C4b2a3b.

21. The method of claim 1, wherein the antibody is of an IgG class.

22. The method of claim 1, wherein the antibody crosses the blood brain barrier.

23. The method of claim 22, wherein the antibody is covalently linked to a therapeutic agent.

24-27. (canceled)

28. The method of claim 1, wherein the antibody specifically binds to and neutralizes a biological activity of C4.

29. The method of claim 28, wherein the biological activity is 1) C2 binding to C4b, (2) C4 cleavage by C1s, (3) C4 cleavage by MASP-1, (4) C4 cleavage by MASP-2, (5) C4 cleavage by MASP-1 and MASP-2, (6) C2a binding to C4b, (7) C4b binding to or cleavage of C3 (as part of the C3 convertase), or (8) C2a binding to or cleavage of C5 (as part of the C5 convertase).

30. The method of claim 28, wherein the biological activity is (1) neutralization of the classical complement activation pathway, (2) neutralization of the lectin complement activation pathway, (3) neutralization of the alternative pathway activity, (4) neutralization of the membrane attack complex (MAC), (5) activation of antibody and complement dependent cytotoxicity, (6) CH50 hemolysis, (7) synapse loss, (8) B-cell antibody production, (9) dendritic cell maturation, (10) T-cell proliferation, (11) cytokine production, (12) microglia activation, (13) Arthus reaction, (14) phagocytosis of synapses or nerve endings, or (15) activation of complement receptor 3 (CR3/C3) expressing cells.

31-33. (canceled)

34. The method of claim 1, wherein the antibody is a monoclonal antibody, a polyclonal antibody, a recombinant antibody, a humanized antibody, a chimeric antibody, a multispecific antibody, or an antibody fragment thereof.

35. The method of claim 34, wherein the antibody is an antibody fragment and the antibody fragment is a Fab fragment, a Fab′ fragment, a F(ab′)₂ fragment, a Fv fragment, a diabody, or a single chain antibody molecule.

36-37. (canceled)

38. A method of inhibiting synapse loss comprising administering to a patient suffering from adverse synapse loss an antibody as defined in claim 1.

39-50. (canceled)

51. A method of treating or preventing a disease associated with complement activation in an individual in need of such treatment, the method comprising administering the antibody of claim 1.

52-66. (canceled)
